(19) United States
(12) Patent Application Publication
(10) Pub. No.: US 2010/0229879 A1
(43) Pub. Date: Sep. 16, 2010

(54) METHOD OF TREATMENT OF INSULIN RESISTANCE SYNDROME (IRS)

(76) Inventor: Watve MILIND, Maharashtra (IN)

Correspondence Address: LUCAS & MERCANTI, LLP, 475 PARK AVENUE SOUTH, 15TH FLOOR, NEW YORK, NY, 10016, US

(21) Appl. No.: 12/556,829

(22) Filed: Sep. 10, 2009

(30) Foreign Application Priority Data
Sep. 12, 2008 (IN) 1942MUM2008

(51) Int. Cl. A61B 17/00

(52) U.S. Cl. 128/898

(57) ABSTRACT

The present invention relates to a method of treatment of Insulin Resistance Syndrome (IRS), said method comprising the following steps:
i. increasing secretions of hormones and growth factors by physically aggressive activities;
ii. normalizing monocyte and neutrophil count by cutaneous immuno-sensory stimulation;
iii. stimulating wound healing and angiogenesis mechanisms by physically aggressive activities and skin stimulation exercises;
iv. stimulating insulin secretion by intense mental activity and by increased epidermal growth factor (EGF) production which is stimulated by physically aggressive activities; and
v. decreasing insulin resistance by isolation of the patient in a serene environment.

### FIELD OF THE INVENTION

The present invention relates to the field of metabolic disorders and their treatment. More particularly, the present invention relates to Insulin Resistance Syndrome (IRS) and its treatment by Holistic Behavioral Therapy (HBT).

### BACKGROUND OF THE INVENTION AND PRIOR ART

The Insulin Resistance Syndrome (IRS), also called metabolic syndrome or syndrome X, consists of a cluster of conditions including hyperinsulinemia, insulin resistance, impaired glucose tolerance, type 2 diabetes, hypertension, hypercholesterolemia, atherosclerotic vascular disease, coronary artery disease, retinopathy, nephropathy and impaired wound healing. IRS is one of the major causes of mortality in the modern world. Insulin resistance is a condition in which tissues exhibit reduced response to insulin. This is accompanied by increased insulin synthesis resulting in hyperinsulinemia which is thought to be a compensatory response to reduced insulin sensitivity. A preliminary diagnostic criterion for insulin resistance is high levels of plasma insulin in relation to glucose levels. It is believed that the ability of the pancreatic beta cells to meet the demand posed by progressive insulin resistance ultimately reaches its limits. By the classical interpretation, the failure of beta cells to provide for the increased demand for insulin leads to type 2 diabetes. Obesity is positively correlated with insulin resistance and thought to be a major contributor to it. Many of the pathological effects of this syndrome are now known to be due to a low grade chronic systemic inflammation, angiogenesis dysfunction and oxidative stress.

Insulin resistance was thought to be a tendency evolved to survive periodic starvation in hunter gatherer life. This hypothesis popularly known as “thrifty gene” hypothesis was shown to be inadequate to account for the diverse functions of insulin and the consequences of insulin resistance on the body. This hypothesis and its variations have been challenged recently on several grounds (Speakman et al, Diab Vase Dis Res 2006, Watve et al, BMC Evolutionary Biology, 2007). Watve et al proposed an alternative hypothesis for the evolutionary origins of insulin resistance. According to this hypothesis insulin resistance evolved as an ability to switch over reproductive and life sustenance strategies in one's life time. The two types of transitions that trigger insulin resistance are (i) ‘Soldier’ to ‘diplomat’ lifestyle and (ii) r to K strategy of reproduction. The soldier to diplomat transition refers to a shift from a muscle dependent to brain dependent life style or transition in personality from a fighter-forager to social manipulator.

Theoretical as well as empirical studies have shown that similar to the soldier and diplomat dichotomy in humans, physically aggressive and nonaggressive strategists coexist in animal societies with negative frequency dependent selection. Game theory has shown that an aggressive ‘hawk’ strategy can coexist with a submissive ‘dove’ strategy in Nash equilibrium owing to negative frequency dependence (Maynard Smith, Cambridge University Press, 1982). In empirical studies, although dominant individuals are found to have a higher reproductive success through means such as greater access to females, subordinate individuals have alternative means such as sneak-mating for gaining a substantial reproductive success (Ohsawa et al, Primates, 1993; Rasmussen et al, Journal of Insect behavior, 1994; Pilastro et al, Proc. R. Soc. Lond. B, 1999; Alberts et al, Animal Behaviour, 2006). Differential reproductive success of aggressive and non-aggressive individuals under different socio ecological conditions is also studied in human populations. These studies show that aggressive individuals have greater reproductive success in one set of conditions but non-aggressive have greater reproductive benefits in certain other conditions (Beckerman et al, PNAS, 2009). The concept of differential success of alternative behavioral strategies applies not only to sex and reproduction but also other competitive interests such as access to food and other essential resources. Two distinct classes of coping strategies are demonstrated in rodents in which the aggressive strategists show an active response to stress situations and in situations of defeat they react with flight or escape. On the other hand the non-aggressive strategists are passive and in aversive situations react by immobility and withdrawal rather than flight and escape (Benus et al, cell mol life sci 2005). These alternative behavioural strategies are associated with different neurophysiologic and metabolic states. For example, subordinate males in animal societies typically have low testosterone, high plasma cholesterol and corticosterone (Blanchard et al, Behav Brain Res 1993) and elevated serotonin signaling whereas dominant males are characterized by high testosterone (Blanchard et al, Behav Brain Res 1993), low serotonin (Kaplan et al, Psychosomatic Medicine, 2002) and lower plasma cholesterol (Kaplan et al, Psychosomatic Medicine, 1991). There are many close parallels between the ‘hawk’ and ‘dove’ strategies of animal behavior and ‘soldier’ versus ‘diplomat’ lifestyles in humans. However there are two important differences between ‘doves’ and ‘diplomats’: (i) In animal societies subordinate individuals generally have a lower social status. Patches of richer food are more likely to be snatched by dominant individuals leaving the subordinate ones with less calorie rich food. Since subordinate individuals are likely to have less access to food, they need to develop a “thrifty” metabolism. Occasionally, if a subordinate individual discovers a patch of food, in order to derive maximum benefit from it before attracting the attention of dominant individuals, binge eating would be adaptive. Social subordination is shown to induce high fat intake and weight gain (Moles et al, Psychoneuroendocrinology 2006) and this would be adaptive for individuals who have only occasional access to rich food. Unlike ‘doves’ in animal or ancestral human societies, ‘diplomats’ in modem human societies can have higher social status and continued access to calorie rich food. The high calorie availability for physically week and inactive individuals poses a paradox that is characteristically human and more typical of modern lifestyle. (ii) Physical aggression is the only predominant form of aggression in animals whereas verbal and political aggressions are alternative forms of aggression in humans whose metabolic requirements are likely to be much different than physical aggression. We use the term aggression here to imply physical aggression. Verbal and political aggressions are assumed to be components of ‘diplomat’ rather than ‘soldier’ strategy. Despite the differences, the close parallels between animals and humans in the behavioural dichotomy imply that metabolic adaptations accompanying behavioural shifts speculated by the Watve et al (2007) hypothesis could have arisen very early in vertebrate evolution.

Food and sex are the main natural causes of aggression. However, since aggression increases the risk of injury, aggression control is equally crucial. When there is no desperation for food or sex, aggression should be suppressed. Therefore satiety signals should induce aggression suppression. It is not surprising that the satiety hormone serotonin has a major role in aggression control across vertebrate taxa (Larson et al Behavioural Brain Research, 2001; Ferrari et al, European Journal of Neuroscience, 2003; Chiavegatto et al, Horm. Behay. 2003; Cleare et al, Psychiatry Research, 1997). Serotonin levels in the brain are elevated by metabolic cues of satiety such as carbohydrate consumption or glucose stimulated elevation in insulin levels (Wurtman et al, Obes Res., 1995). Sexual satiety has similar effects on serotonin (Lorrain et al J. Neurosci 1997, 1999). Further chronically elevated serotonin signaling in the hypothalamus has been shown to induce peripheral insulin resistance (Luo et al, Neuroendocrinology, 1998; Luo et al, Neuroendocrinology, 1999; Rattigan et al, Diabetes 1999). This implies that control or suppression of aggression and insulin resistance are endocrinologically linked. Insulin resistance shifts the energy allocation balance from muscle to brain (Watve et al, BMC Evolutionary Biology, 2007) and is thereby a physiologically adaptive response to a soldier to diplomat transition. Furthermore hyperinsulinemia enhances cognitive brain functions (Zhao et al, J. Biol. Chem., 1999; Zhao and Alkon, Mol and Cell Endocrinol, 2001). When the opponent is stronger, aggression is likely to be ineffective or even counterproductive and should be suppressed. Accordingly weak muscles and bones are also predisposing factors to diabetes and any factor increasing muscle or bone strength such as osteocalcin or vitamin D are insulin sensitizing (Chin et al, American Journal of Clinical Nutrition, 2004; Borissova et al Int J Clinical Practice 2003; Ferron et al PNAS 2007, Mc Pherron et al J. Clinical Invest 2002). Hormones associated with physical aggression have a protective role against a number of co-morbidities of the insulin resistance syndrome. On the other hand a number of molecules and mechanisms involved in the control or suppression of aggression enhance obesity and insulin resistance. Physical aggression is known to increase secretion of epidermal growth factor (EGF) in anticipation of injuries (Nexo et al, Acta Physiol Scand., 1981; Nexo et al, Regulatory Peptides, 1984) and EGF is important in wound healing as well as in pancreatic beta cell regeneration (Brand et al, Pharmacol Toxicol, 2002; Bouwens and Rooman, Physiol Rev, 2005). Aggression related hormones also facilitate angiogenesis and angiogenesis dysfunction is the root cause of a number of co-morbidities of insulin resistance syndrome (Ritz et al, Journal of Internal Medicine, 1999; Bishop et al, Endocrine Abstracts, 2008). Reduced injury proneness resulting from non-aggressive lifestyle would also reorient the immune system resulting into delayed wound healing on the one hand and increased systemic inflammation on the other (Watve and Mandani, Curr. Sci. 2008). Diabetes is negatively associated with aggression in rats as well as in humans (Field et al, Journal of Health and Human Behavior 1963; Leedom et al, Pysiol Behav, 1987). It is possible therefore that lack of physical aggression accompanying a soldier to diplomat transition is the major behavioural cue for the development of metabolic syndrome.

This hypothesis implies that insulin resistance is more of a behavioral than a dietary phenomenon. The principle underlying this invention is that if the root cause for the disorder is behavioral, the treatment also should involve a strong behavioral component complimenting and if possible replacing drug treatment. This central concept is the thought process for a method of treatment of metabolic syndrome as envisaged in accordance with this invention.

### OBJECTS OF THE INVENTION

It is an object of the present invention to provide a therapy for metabolic disorders.

It is another object of the present invention to provide a holistic behavioral therapy for Insulin Resistance Syndrome (IRS).

It is still another object of the present invention to provide an effective therapy for Insulin Resistance Syndrome (IRS).

It is yet another object of the present invention to provide a safe therapy for Insulin Resistance Syndrome (IRS).

It is yet another object of the present invention to provide a therapy for Insulin Resistance Syndrome (IRS) which is free of side effects.

### SUMMARY OF THE INVENTION

In accordance with the present invention there is provided a method of treatment of Insulin Resistance Syndrome (IRS), said method comprising the following steps:
i. increasing secretions of hormones and growth factors by physically aggressive activities;
ii. normalizing monocyte and neutrophil count by cutaneous immune-sensory stimulation;
iii. stimulating wound healing and angiogenesis mechanisms by physically aggressive activities and skin stimulation exercises;
iv. stimulating insulin secretion by intense mental activity and by increased epidermal growth factor (EGF) production which is stimulated by physically aggressive activities; and
v. decreasing insulin resistance by isolation of the patient in a serene environment.

Typically, the hormones stimulated by physically aggressive activities are testosterone, estradiol, dopamine, melatonin and cholecystokinin

Typically, the growth factor is epidermal growth factor (EGF).

Typically, the physically aggressive activities involve games and activities which mimic neuromotor actions of stone age hunters while performing acts like hunting, fighting, punching, hard hitting, aiming, throwing, grabbing, snatching, dodging, escaping, chasing and catching.

Typically, the cutaneous immuno-sensory stimulation is induced by pricking, scratching, pinching, and bruising without causing overt injuries.

Holistic Behavioral Therapy (HBT) in accordance with the present invention targets four pathological factors in the following order of emphasis:
1. Systemic inflammation: the systemic inflammatory response is presumed to be an effect of immune reversal following a non-injury-prone lifestyle. HBT attempts to reverse this condition by engaging the patients in acts and games of physical aggression. When the body performs aggressive acts such as hitting or aiming and throwing, the brain predicts increased probability of injury owing to retaliation and prepares to send the immune system peripheral. Also a specifically designed devise for cutaneous immuno-sensory stimulation (CISS), and activities exposing the skin to non-pathogenic microorganisms are used to attract immune cells towards the skin. This is expected to reduce immune cell density in blood vessels and increase that in peripheral tissues. The normalization of immune response also results in reduction in oxidative stress since the immune cells are the major sources of reactive oxygen species.
2. Angiogenesis and would healing mechanisms: A non-injury-prone lifestyle leads to disinvestment from wound healing and angiogenesis mechanisms. Aggressive games and CISS are used to stimulate angiogenesis as well as wound healing mechanisms.
3. Beta cell function and insulin secretion: Insulin is needed for many cognitive functions of the brain and intensive brain activation is expected to increase insulin secretion. Also since there is evidence that beta cell damage is cytokine mediated, reduction in systemic inflammation is expected to enhance beta cell regeneration. Aggression increases EGF levels in saliva and plasma and this stimulates beta cell regeneration in addition to improving wound healing. Therefore a combination of brain exercises and physical aggression would enhance beta cell regeneration and insulin secretion.
4. Insulin resistance: reducing insulin resistance is the last and relatively low priority target of HBT since insulin resistance by itself may not be bad for health in the absence of immune reversal, angiogenesis dysfunction and beta cell damage. Exercises and activities for decreasing IR include solitude, developing natural history as a hobby, meditation, agility and reflex exercises as well as physical aggression.

### BRIEF DESCRIPTION OF ACCOMPANYING DRAWINGS

The invention will now be described with reference to accompanying drawing in which:

FIG. 1 shows a polymer ball specifically designed for HBT; (A) Cut Ball Views and (B) Cut-Ball isometric projections.

FIG. 2 shows orthographic views of a CISS Cuff (a) Front view, (b) Right hand side view and (c) Top view.

FIG. 3 shows a normalizing action on HbAlc which is implied by the significant negative correlation between initial level and change after treatment.

FIG. 4: shows mean and standard error of level of HbAlc before and after treatment for patients whose pre-treatment levels were above 7%.

FIG. 5: shows a normalizing action of HBT on peripheral blood leucocyte density

FIG. 6: shows a normalizing action of HBT on granulocyte density

FIG. 7: shows a normalizing trend of inflammatory marker C reactive protein (CRP)

FIG. 8: shows mean and standard errors of pre and post treatment C reactive protein (CRP) levels for patients whose pre-treatment CRP levels were above normal

FIG. 9: shows negative correlation of change in fasting plasma insulin after HBT with pre treatment HOMA R

FIG. 10: shows mean and standard errors of pre and post treatment of an index of beta cell function (Homa beta)

FIG. 11: shows mean and standard errors of pre and post treatment of an index of insulin resistance (Homa R)

### DETAILED DESCRIPTION OF THE INVENTION

In accordance with the present invention there is provided a Holistic Behavioral therapy (HBT) for the treatment of Insulin Resistance Syndrome (IRS). The method of treatment in accordance with this invention consists of reducing the stimuli identified mainly by the behavioral switch hypothesis that trigger the development of insulin resistance and related morbidities. The stimuli identified and targeted here are as follows.
1. Exposure to crowding: Chronic exposure to crowding has been shown to reduce physical aggression (Judge et al, Animal Behaviour, 1997). Isolation increases aggressive responses in rats (Wongwitdecha et al, Behavioural Brain Research, 1996). Optimum reproductive strategies also change according to the population density. At high population densities a K strategy is more appropriate than r strategy and since insulin resistance causes a shift from r to K, according to the Watve-Yajnik hypothesis, high population density is expected to trigger insulin resistance. There is epidemiological evidence that people living in more crowded places have a greater risk of getting diabetes, although this had not been hitherto interpreted as an effect of population density (Ellaway et al, Br Med 3, 2005, Riste et al, Diab Care, 2001). The epidemic of metabolic syndrome is likely to be a response to the increasing population density as pointed out by Watve and Yajnik. Exposure to crowding is an individual's sampling of the global polulation density and his behavioral strategies get modified accordingly.
2. Lack of physical strength, activity, agility, aggression and defense reflexes: In traditional concept of control of the IRS, physical exercises are of prime importance. However, exercises are traditionally viewed as means of burning calories. According to the behavioral switch hypothesis, the role of exercises is much beyond calorie burning. According to the our hypothesis, weak muscles and bones, slowing down of reflexes and loss of physical aggression are indicators of a ‘diplomat’ mode of life and therefore are risk factors for type 2 diabetes. Insulin is shown to be involved in fine manipulation of pre-attentive versus attentive neural responses and hyperinsulinemia is a primary response of the body to the soldier to diplomat transition.
3. The need for and skills of social manipulation: A diplomat mode of life needs social manipulation skills. There is a trade-off between physical strength and social manipulation skills. Physical actions that mimic retreat facilitate cognitive functions of the brain (Koch et al, Psychological Science, 2009). Therefore individuals whose nature or profession demands social manipulation skills are at a higher risk of developing some components of metabolic syndrome.
4. Stress related to social hierarchy: Social hierarchy has a very crucial role in the behavioral switch hypothesis. Social hierarchies are shown to be important determinants of metabolic disorders (Sapolsky Science 2005).
5. Loss of sexual desire and activity: There is a progressive deterioration of sexual desire and sexual function in IRS. The traditional interpretation has been that insulin resistance leads to loss of sexual function. However, some recent work has shown that improved glycemic control does not improve sexual functions but long term treatment for sexual dysfunction improves insulin sensitivity (Ayala et al., Diabetes, 2007). It is logical therefore that improved sexual activity has a therapeutic effect on metabolic syndrome.
6. Obesity: Obesity makes quick physical actions difficult owing to which obese individuals are unlikely to be successful in ‘soldier’ lifestyle and are forced to undertake ‘diplomat’ life. CBI receptor molecules of the endocannabinoid system that play a role in obesity are involved in aggression control (Martin et al, Psychopharmacology, 2002). It is well known that obesity induces insulin resistance by a variety of mechanisms. Also adipokines are responsible for immune redistribution characteristic of type 2 diabetes (Watve et al, Curr. Sci., 2008).
7. Lack of immune-stimulation of the skin in the absence of injury proneness: In a soldier life skin injuries of varying magnitude are expected to be more common. Therefore the immune system is driven to the subcutaneous tissues. On shifting to a diplomat life, the immune system is withdrawn from the periphery. This leads to delayed wound healing on the one hand and increased inflammation proneness of central tissues on the other. This immune reversal, i.e. retracting the immune system from the subcutaneous tissues is a major contributor to the pathological consequences. Immunity related changes in the body are increasingly being known as the cause of many of the pathological effects of IRS. Also the non-injury-proneness leads to disinvestment from angiogenesis and wound healing mechanisms and angiogenesis dysfunction is the major cause of many pathological components of the metabolic syndrome.

The method of treatment in accordance with this invention (HBT) targets all the above risk factors and attacks the behavioral root cause behind metabolic syndrome. Some of the components of HBT are well known and practiced by some or the other of the alternative schools of medicine. There are many others which are new concepts and unique to HBT. Modern medicine emphasizes on reducing obesity, reducing insulin resistance and administration of insulin. Low calorie diet and calorie burning exercises are also emphasized. Yoga emphasizes on stress relief and body flexibility. Acupuncture and related therapies mobilize the immune system towards the periphery and thereby reduce systemic inflammation. HBT is a combination of all the above in addition to its unique components and is the first system to address all the causes and stimuli for the development of metabolic syndrome together.

The HBT in accordance with this invention has many novel components such as solitude, aggressive games and exercises, exercises for agility, swiftness and sharpening reflexes, exercises for immuno-sensory stimulation of the skin and mental exercises to stimulate insulin secretion. These components act holistically and synergistically.

A HBT protocol in accordance with this invention comprises the following components:

### Environmental Components:

1. According to the behavioral switch hypothesis population density is a strong stimulus for development of insulin resistance. Since population density has been implicated as a trigger to develop insulin resistance, in order to reduce its influence or counterbalance its effects, periodic retreat to solitude and a serene environment is advocated in HBT. This involves an exposure to and concentrating on open expanses, the skies, seas, hills and forests. During the treatment period participants are made to go alone and sit all alone in a place away from any other human activities and spend about an hour at a time in a serene environment. A mental experience of serenity is more crucial than physical presence in a serene place. This is inculcated by a basic training in natural history and developing a habit of observing birds, butterflies, flowering plants or any such forms of life around.
2. Participants are advised and instructed to grow greenery around their residence and workplace to a maximum possible extent. Training and advice is provided on the best ways to grow terrace gardens, kitchen gardens and the like.
3. This component also includes yoga, meditation, painting, landscaping, poetry or music which are alternative ways of getting a feeling of tranquility.

### Physical Components:

1. Exercises specially designed for the following are included in an optimized combination in HBT.
   a. Muscle and bone strength: These include power exercises such as push-ups or sit-ups, exposure to sunlight as to enhance vitamin D synthesis.
   b. Physical aggression: A major component of HBT is games and activities mimicking neuromotor actions in stone age hunting or fighting e.g. punching, hard hitting, aiming and throwing, chasing, catching on the one hand and dodging, escaping etc on the other. A predominant hypothesis behind HBT is that when one makes physically aggressive actions the brain predicts retaliation and therefore possible injuries to the skin. Therefore aggressive actions stimulate (i) production of epidermal growth factor (EGF) useful for wound healing. Since EGF also facilitates pancreatic beta cell regeneration, aggressive actions help normalize insulin synthesis and secretion in the long run. (ii) stimulation of red blood cell synthesis by enhanced erythropoietin synthesis in anticipation of blood loss through injuries (iii) activation of angiogenesis mechanisms in anticipation of injuries and (iv) mobilization of immune cells towards the skin. All these effects of physical aggression are desirable in management and treatment of type 2 diabetes.
   c. Form, figure and flexibility: e.g. aerobics and yoga
   d. Endurance: e.g. brisk walking, jogging and swimming
   e. Agility and fine-tuning of motor reflexes: e.g. fast games. Quick actions in response to sudden unpredictable situations are important for sharpening reflexes. For example these exercises would include simple ball games such as catching or dodging but where there is certain unpredictability about the position and momentum of the ball and quick judgment and fast action is necessary. A number of such exercises and games are designed for HBT.

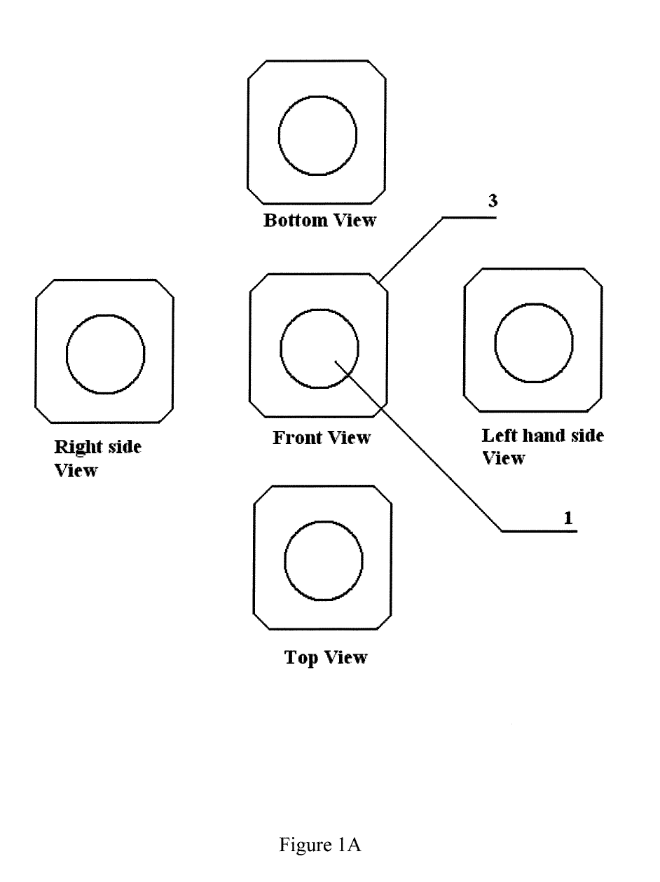

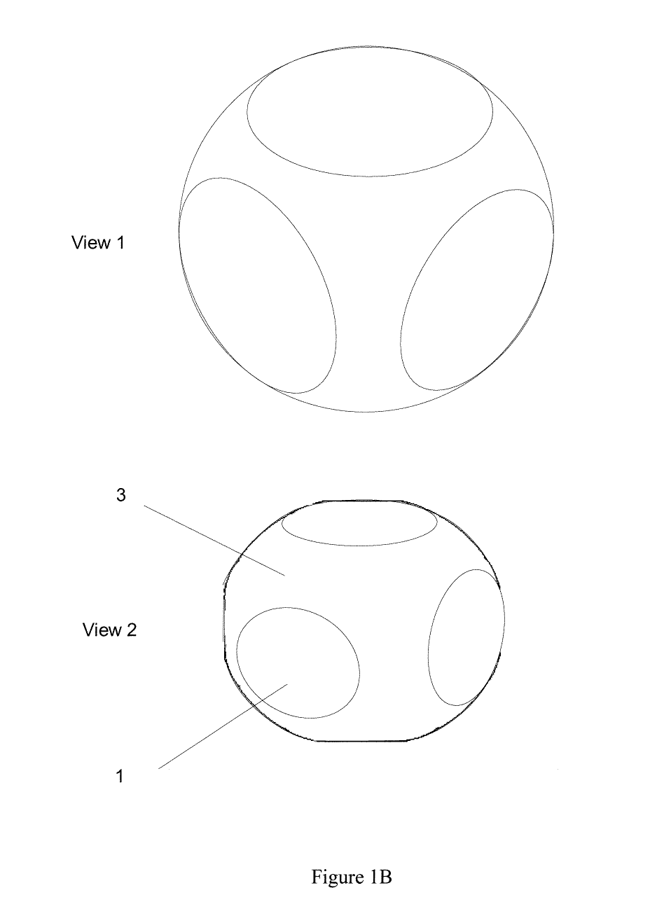

A device specifically designed for HBT consists of a ball made up of a polymer giving good bounce. The ball is molded or cut or machined such that it has one or more flat surfaces and many edges and angles. An example is shown in FIG. 1). The flat surfaces (3) or edges (5) make the bounce unpredictable and necessitates quick actions to catch or dodge the ball. A variety of games can be devised with a cut ball that can be played by a single person, in pair or in a group. For example a single person can stand at a specific distance from a wall, bounce the ball against the wall and catch it. The difficulty level of the exercise can be varied by changing the distance from the wall, the speed and angle of throw or adding one bounce on the ground. Two or more people can play a table tennis like game across a table or on plane ground. Apart from physical exercise, these games have a possible mental effect. A hypothesis in HBT is that when the brain feels the need for agility and swift action, it regulates the distribution of body fat to enhance agility. This is expected to result in reduced abdominal obesity which is a major risk factor for metabolic syndrome.
2. A treatment system involves mobilizing the immune system back towards subcutaneous tissues. Increasing skin exposure to soil and other natural sources of nonpathogenic bacteria is crucial. This is achieved by traditional farming and gardening activities as well as creative work with earthenware on a potter's wheel. This is intended to divert the immunological apparatus towards the body surface, away from internal organs.
3. Diabetes patients have a compromised wound healing system. Therefore in traditional treatment they are advised to avoid any kind of injurious exposure of the skin. This is likely to enhance the immune reversal further, making the cutaneous immune system further weaker. Recent research has also demonstrated that cutaneous nerves and neuropeptides are active immunomodulators. In diabetes there is progressive degeneration of peripheral nerves, further deteriorating the cutaneous immune system. Simple skin stimuli have been shown to activate the immune cells. Therefore, HBT involves exposure to subtle cutaneous challenges such as pricking, scratching, pinching and bruising without causing overt injuries. This is achieved by walking barefoot on carefully prepared tracks that will stimulate the foot skin without resulting into any penetrative injuries, sitting and sleeping on surfaces prepared from grass, bamboo, sand and such material of a kind of consistency that will not cause any penetrative skin damage but cause subtle pricking and pinching stimuli.

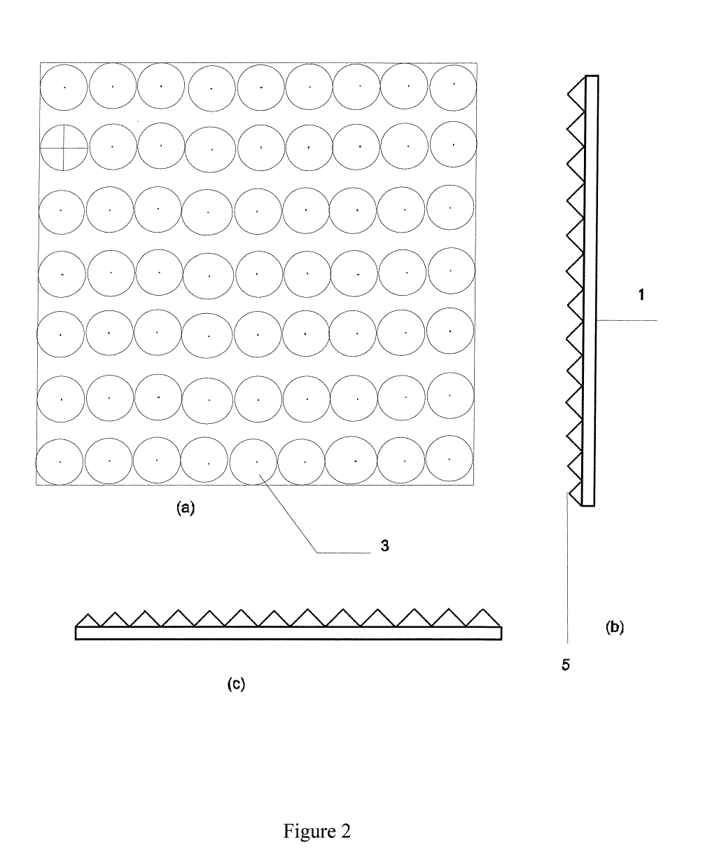

A device is specially designed for non-penetrative cutaneous immuno-sensory stimulation (CISS) as shown in FIG. 2). The device consists of a strip (1) made up of soft wood, rubber, polymer or any suitable material having multiple pointed projections (3), an inflatable cuff such as the one used for blood pressure measurement and a rubber bulb to inflate the cuff. The strip (1) is wound around any portion of the arm, thigh or feet such that the pointed projections (3) rest on the skin The inflatable cuff is wound around it and inflated by using rubber bulb. The patient is asked to concentrate on the pricking pain and as the pressure in the cuff is increased and report when the pain becomes intense. This pressure is maintained for 30 seconds after which air is let out and the device is removed. This CISS procedure may be repeated up to 3 times in a day on different parts of skin.
4. Sensory exercises: The patients are first asked to close their eyes or blinded by any suitable device. A trainer or helper then touches the patient's skin in one or more places with a soft device such as peacock's feathers and the patient is asked to identify the places being touched. In an alternative game, a number of small marbles are tucked in a slab of clay, plasticine or such suitable material in any specific formation and patients are asked to touch the formation with different parts of the skin such as elbow, toe or back of the arm and identify the formation as well as count the number of marbles. This is intended to stimulate the sensitivity and regeneration of peripheral sensory nerves.
5. The fat stores of the body respond to temperature cycles. There is more subcutaneous fat deposition in cold climate which is disposed by the body rapidly in warm climates. Natural temperature cycles keep the fat tissue mobilized whereas modern life style with temperature regulated homes, cars and working places results into greater stagnation of fat tissue. Exposure to the daily natural temperature cycles during HBT training and advice to minimize the use of air-conditioner is a part of the treatment.
6. The HBT protocol includes games involving hunting instincts. For example catching fish by hand from a tub or pond of water. This needs patience, concentration, reflexes and quick action-typical traits of a skilled hunter.
7. Testosterone, a hormone involved in male aggression is known to give some degree of protection against metabolic disorders. A psychological feeling of a “soldier” life is just as important as physical participation in aggressive games and acts. The soldier' exercises not only include punch bag, archery and darting but also include imagination games and training for self defense. For example, in a musical chair like game a number of possible dangers are displayed on a screen such as, dangerous animals, air-raid, earthquake or flood. Players are previously instructed to take the right defensive action for each danger. With a beating music players go round in a circle and when the music stops they look at the screen, identify the danger and take the necessary action quickly. The slowest or wrong actor is eliminated. Apart from physical activity the mental exercise of being alert and prepared for self defense is a desired element that is expected to affect the immune system in a way similar to the effects of physical aggression described above.

### Neuro-Psycho-Social Components:

1. Money is a very recent phenomenon in the evolutionary history of humans and therefore separate brain centers to handle money related emotions and information processing are unlikely to have evolved. The brain areas involved in handling food related emotions and information were exapted to handle money. Therefore there could be a cross talk between the neural mechanisms of handling money and food. It's known that the region of the orbitofrontal cortex involved in processing food rewards is also involved in processing money rewards. Briers et al (psychological science 2006) showed that under experimentally manipulated situations hunger affects money related decisions and the desire for money increases hunger. It is also possible that the desire to accumulate wealth results into a tendency to store fat. Therefore attitude towards money and wealth needs to be modified. A transition from individual possessiveness to trusteeship could be beneficial and this is inculcated through lectures, participatory discussions, imaginary economic games where participants are encouraged to donate and spend money rather than accumulate, counseling and advice.
2. Social hierarchy has a very crucial role in the behavioral switch hypothesis. An experiment in primates showed that social hierarchies are important in development of metabolic disorders. Through lectures, participatory discussions, games, counseling and advice HBT tries to develop attitudinal indifference towards prestige and social status. The cultural atmosphere during HBT training is completely egalitarian. There are no ‘leaders’ in the set-up. All work, including sundry and menial jobs at the camp site are shared equally between the members. For the duration of the treatment, the members are encouraged to ‘forget’ the designations held in their normal life. For example, a simple norm is that everyone is called by his or her first name irrespective of age, prestige and position.
3. Under a state of insulin resistance peripheral tissues such as muscle utilize less amount of glucose and more is made available for the brain. The brain is also an energy intensive organ. An active brain utilizes large quantities of glucose. Therefore mental exercises stimulating memory and thinking are as important as physical exercises. HBT includes many exercises such as Sudoku, crossword and other types of puzzles and will suggest ongoing exercises for follow-up. HBT also encourages performing art and other creative activities. It is known that insulin facilitates cognitive brain activities and mental challenges stimulate insulin secretion. The different memory and thinking exercises of HBT are intended to enhance insulin secretion by the pancreas.
4. Sex life and sex hormones have some degree of protective effects against many components of the IRS. Having active sex in real life is often circumstances driven and beyond individual control. However, mental sex life in the form of sexual fantasies is a natural phenomenon. This deteriorates with age and particularly rapidly in IRS. Promoting an active mental sex life through virtual games, counseling and advice is a part of HBT. For example in an imagination game the patients are shown a series of pictures of the opposite sex and asked to choose a short term or long term sex partner from them with an attempt to identify the features based on which the decision was made. This leads to greater mental involvement in the game and helps stimulate the secretion of sex and aggression related hormones.
5. Stress tolerance: Although stress is often implicated as a predisposing and aggravating factor for many disorders, there is no clear definition and classification of stress. The HBT classifies stress and anxiety as “soldier stress” and “soldier anxiety” versus “diplomat stress” and “diplomat anxiety”. Soldier anxiety is one that predicts physical fights with rivals, predator attacks, physical injuries or discomfort, whereas diplomat anxiety is one that predicts change in social status, financial problems or strained relations. Solutions for situations leading to soldier anxiety involve intense physical action whereas solutions to the causes of diplomat anxiety involve social manipulation. As on the one hand “diplomat anxiety” is a risk factor for IRS, “soldier anxiety” can be protective. Therefore the ill-effects of diplomat anxiety can be balanced by soldier anxiety. Games and activities are designed to inculcate a sense of adventure which is defined as performing an act by overcoming long standing fear. The nature of the act may differ from person to person. For example handling animals like gecko, cockroach or a non poisonous snake can be a good mental adventure for some. Such mental adventures can counter the effects of diplomat anxiety. The HBT philosophy believes that stress is a natural phenomenon and optimum combination of soldier and diplomat stress is beneficial for health. HBT intends to increase stress tolerance and optimize the relative effects of soldier and diplomat stress stress rather than avoid stress which is often impossible in real life.
6. Spatial working memory: Spatial working memory in the form of mental maps and subconscious directional orientation is frequently and intensively used in a hunter gatherer life. These mental abilities are seldom used in modern life and therefore weakened or lost. Elevated serotonin levels in diabetes are associated with loss of spatial working memory. Games are designed to sharpen spatial working memory such as blindfolding and finding the direction after rotating around oneself, blinding and jogging in one place without changing the orientation and position, blinding and listening to a sound from two different positions and locating the source of the sound by triangulation etc.
7. Neuro-motor coordination: games to improve neuromotor coordination typically consist of blindfolding and throwing a ball with one hand and catching with the other hand. The information about the direction and speed of the throw needs to be passed on from one hand to the brain to work out the possible trajectory of the ball and accordingly coordinate the movements of the other hand to catch the ball. The difficulty levels of the game can be adjusted by increasing the distance between the starting positions of two hands and the angle of throw.

### Dietary Component:

A balanced, nutritious, diverse and tasty diet is advocated. Low fat and low calorie diet has failed to reduce obesity and diabetes on an epidemiological scale. Drastic weight reduction programs are counterproductive in the long run and are discouraged by HBT.

In accordance with the present invention there is provided a method of treatment of Insulin Resistance Syndrome (IRS), said method comprising the following steps:
i. increasing secretions of hormones and growth factors by physically aggressive activities;
ii. normalizing monocyte and neutrophil count by cutaneous immuno-sensory stimulation;
iii. stimulating wound healing and angiogenesis mechanisms by physically aggressive activities and skin stimulation exercises;
iv. stimulating insulin secretion by intense mental activity and by increased epidermal growth factor (EGF) production which is stimulated by physically aggressive activities; and
v. decreasing insulin resistance by isolation of the patient in a serene environment.

Typically, the hormones stimulated by physically aggressive activities are testosterone, estradiol, dopamine, melatonin and cholecystokinin

Typically, the growth factor is epidermal growth factor (EGF).

Typically, the physically aggressive activities involve games and activities which mimic neuromotor actions of stone age hunters while performing acts like hunting, fighting, punching, hard hitting, aiming, throwing, grabbing, snatching, dodging, escaping, chasing and catching.

Typically, the cutaneous immuno-sensory stimulation is induced by pricking, scratching, pinching, and bruising without causing overt injuries.

Holistic behavioral therapy in according with the present invention targets four pathological factors in the following order of emphasis:
1. Systemic inflammation: the systemic inflammatory response is presumed to be an effect of immune reversal following a non-injury-prone lifestyle. HBT attempts to reverse this condition by engaging the patients in acts and games of physical aggression. When the body performs aggressive acts such as hitting or aiming and throwing, the brain predicts increased probability of injury owing to retaliation and prepares to send the immune system peripheral. Also a specifically designed devise for cutaneous immuno-sensory stimulation (CISS), and activities exposing the skin to non-pathogenic microorganisms are used to attract immune cells towards the skin. This is expected to reduce immune cell density in blood circulation and increase that in peripheral tissues. The normalization of immune response also results in reduction in oxidative stress since the immune cells are the major sources of reactive oxygen species.
2. Angiogenesis and wound healing mechanisms: A non-injury-prone lifestyle leads to disinvestment from wound healing and angiogenesis mechanisms. Aggressive games and CISS are used to stimulate angiogenesis as well as wound healing mechanisms.
3. Beta cell function and insulin secretion: Insulin is needed for many cognitive functions of the brain and intensive brain activation is expected to increase insulin secretion. Also since there is evidence that beta cell damage is cytokine mediated, reduction in systemic inflammation is expected to enhance beta cell regeneration. Aggression increases EGF levels in saliva and plasma and this stimulates beta cell regeneration in addition to improving wound healing. We therefore expect that a combination of brain exercises and physical aggression would enhance beta cell regeneration and insulin secretion.
4. Insulin resistance: reducing insulin resistance is the last and relatively low priority target of HBT since insulin resistance by itself may not be bad for health in the absence of immune reversal, angiogenesis dysfunction and beta cell damage. Exercises and activities for decreasing IR include solitude, developing natural history as a hobby, meditation, agility and reflex exercises as well as physical aggression.

In accordance with another preferred embodiment of the present invention, no change in diet or total energy expenditure is prescribed during the treatment. Further, there are no immediate changes prescribed in the current medication of any patient but as the blood parameters show improvement medication can be withdrawn in a stepwise manner accompanied by periodic monitoring.

### Pilot Scale Trial:

A pilot scale trial was conducted to test whether HBT has demonstrable effects in the predicted direction. Since the expected effects of HBT are normalizing, the trial and its underlying statistical model needs to be different than conventional clinical trials. In conventional clinical trials the test group is expected to show a significant increase or decrease in a measurable parameter as compared to an appropriate control group. For HBT, since a normalizing action is expected a test for a statistically significant negative correlation between the pre-treatment levels of a parameter and the change in it as a result of treatment was carried out. The regression line should cross the X axis at or near the optimum healthy level of the parameter. Such a correlation would reflect that a parameter value above normal shows a decline and the ones below normal show an increase.

The study was conducted on 30 volunteering participants, 4 of the group were non-diabetic, 6 were type 2 diabetic with good glycemic control (HbAlc<7%) and 20 were type 2 diabetic with poor glycemic control (HbAlc>7%). All participants attended one of the three residential HBT training camps of four days duration each, held between February 2008 and February 2009. The pre treatment blood parameters were analyzed and follow up blood samples were collected after the camp and after 1 and 3 months follow up.

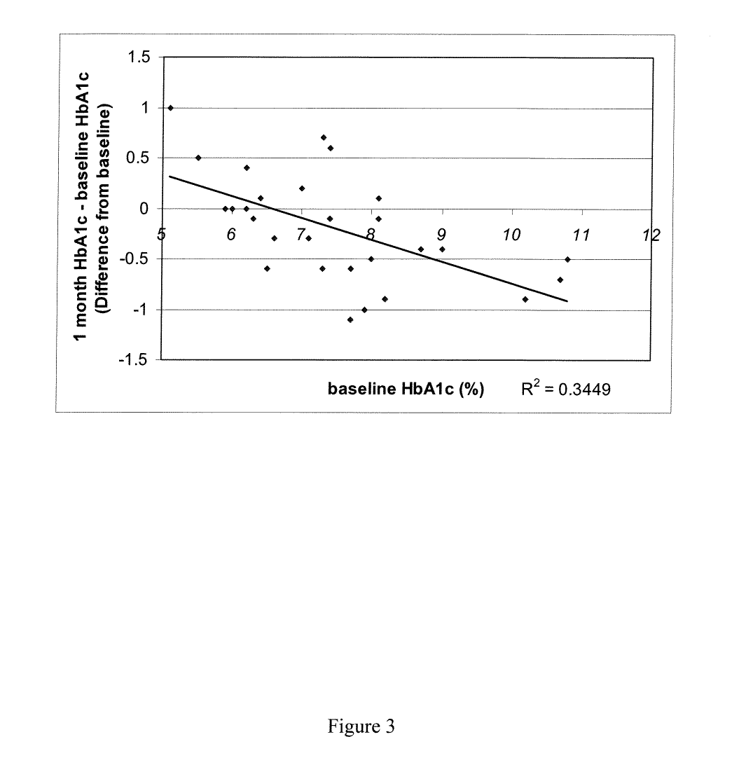

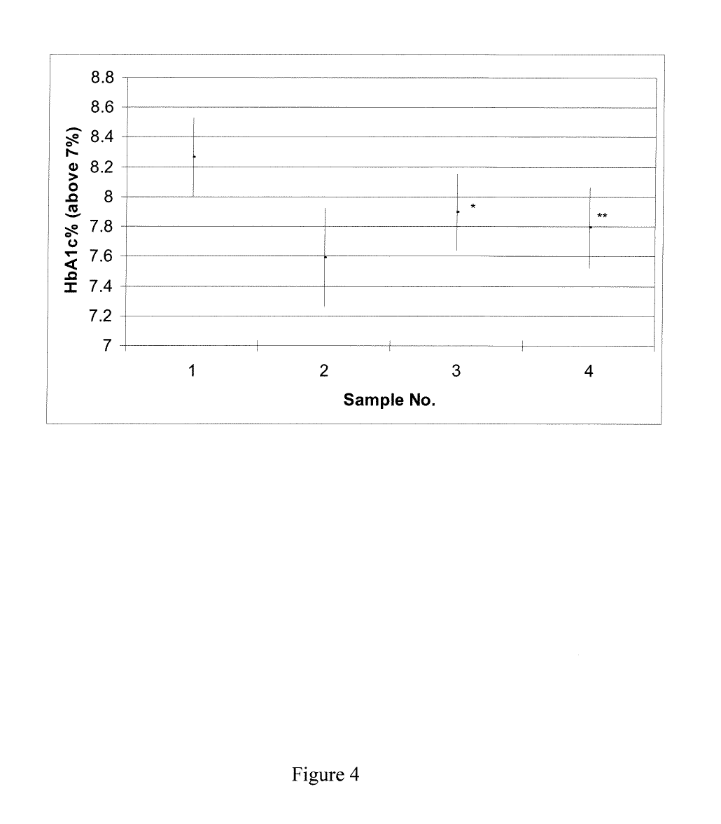

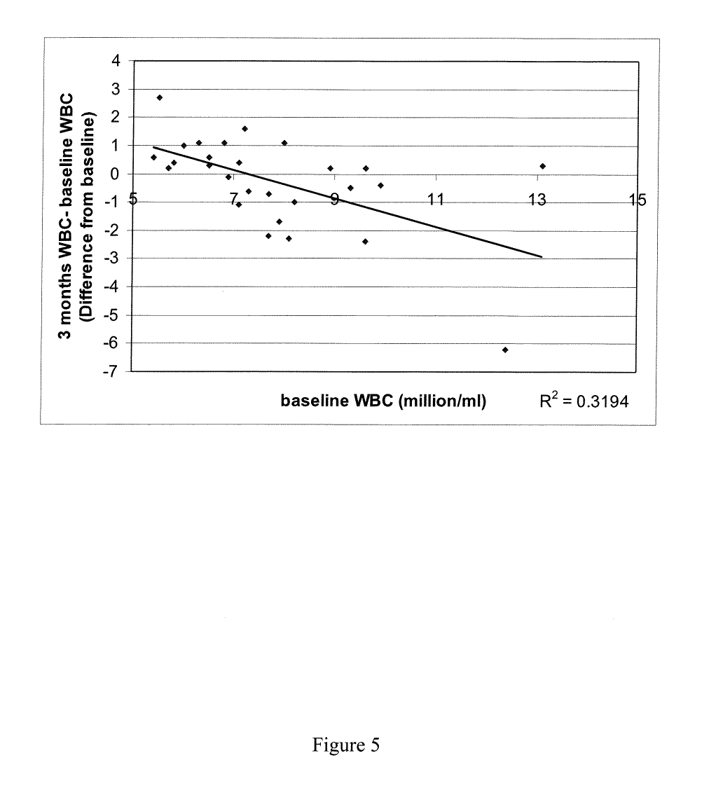

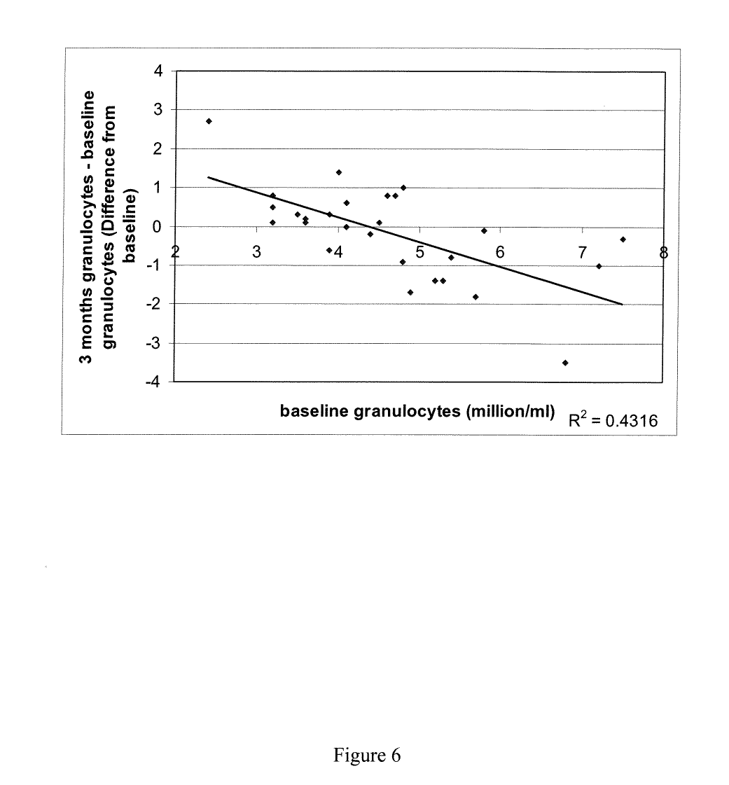

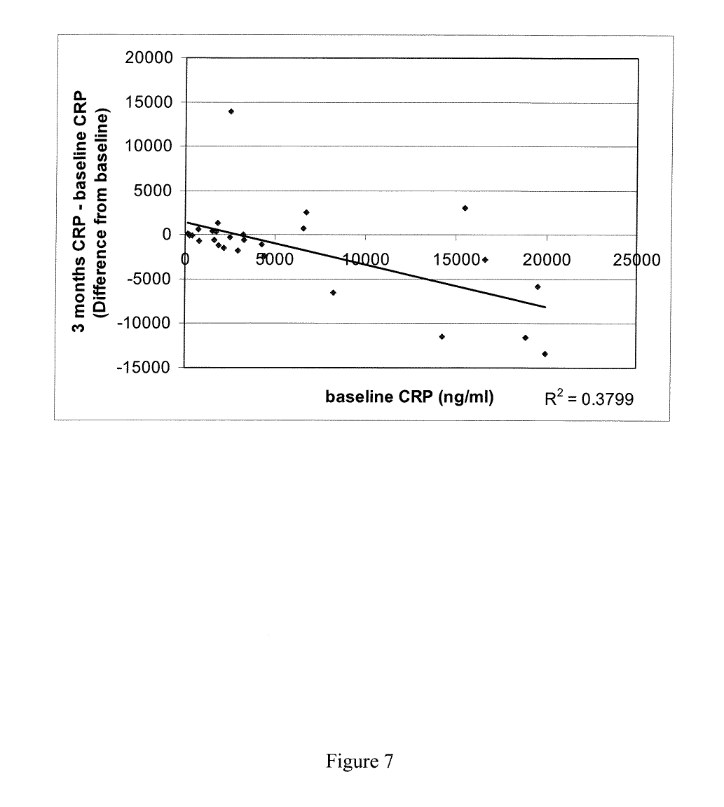

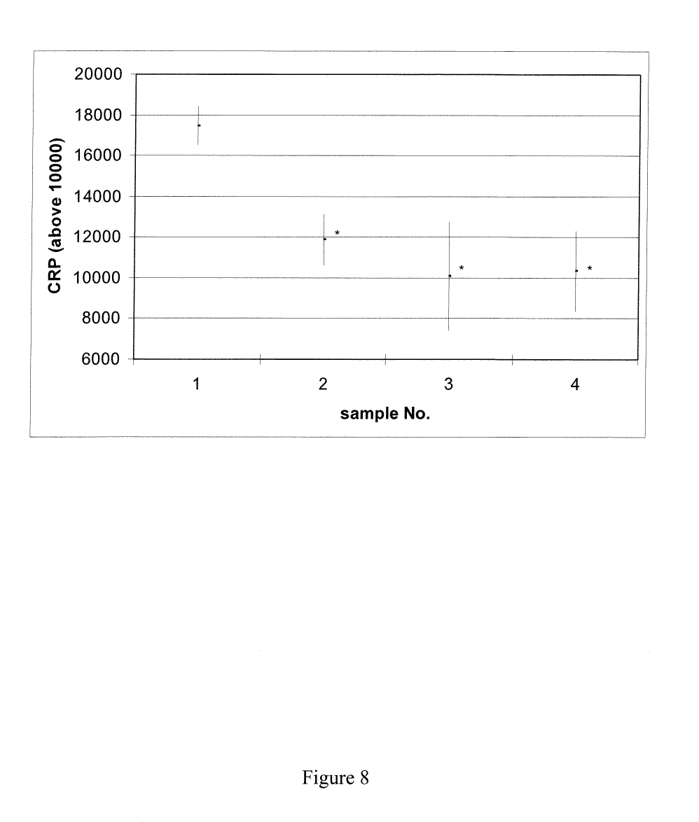

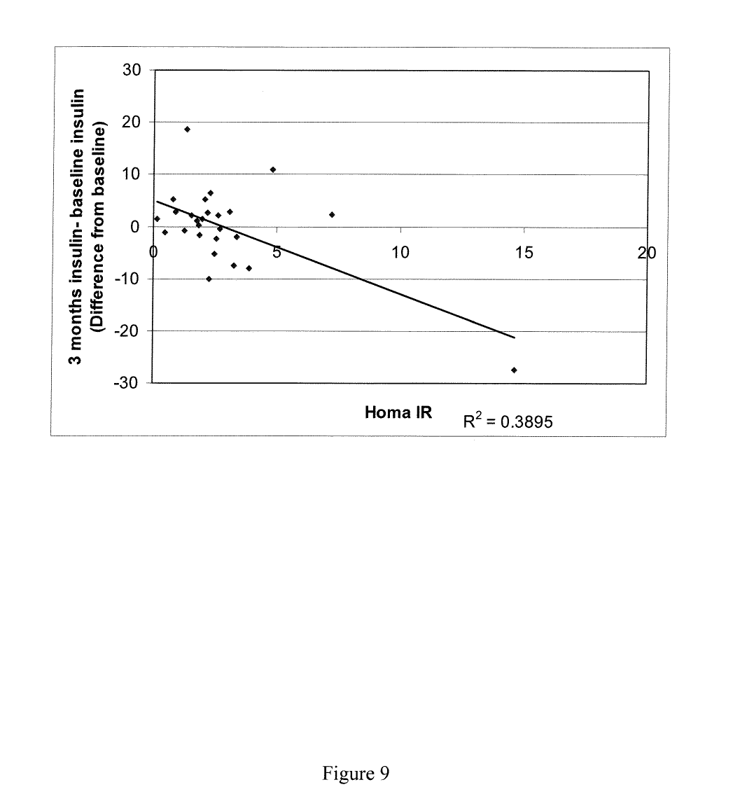

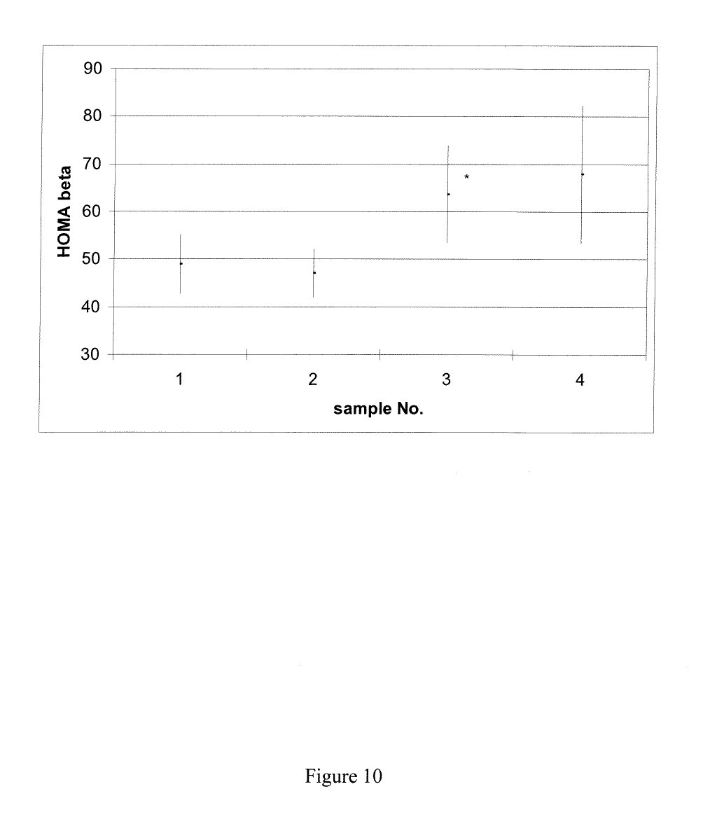

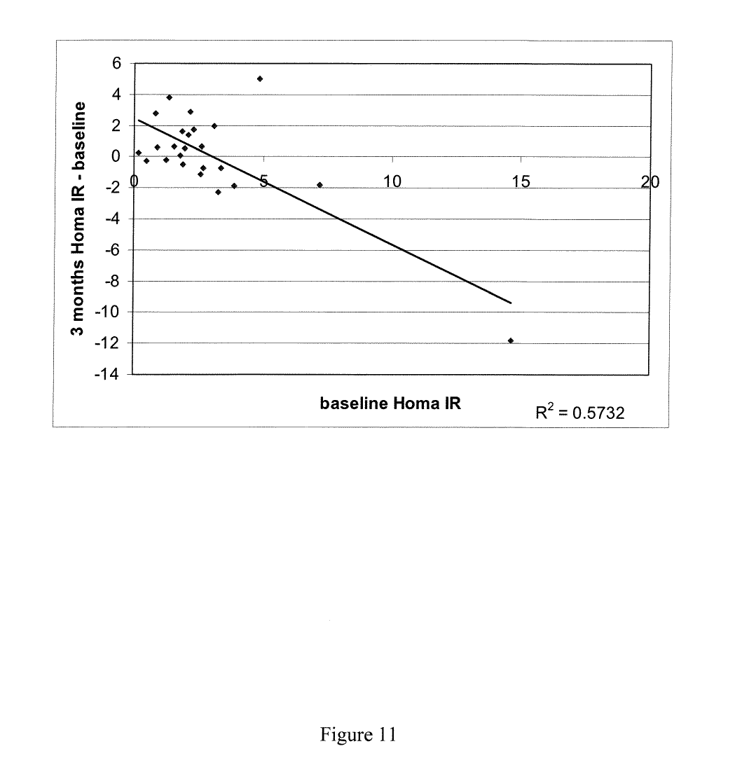

The results of the study are as follows:
1. Glycemic control: The most stable indicator of glycemic control is glycosylated hemoglobin (HbAlc). A normalizing action on HbAlc was implied by the significant negative correlation between initial level and change after treatment illustrated in FIG. 3. Those with initial HbAlc greater than 7 showed a decrease and those with HbAlc less than 6.5 showed an increase in the levels as a result of treatment. A low HbAlc in diabetic patients can be due to frequent bouts of hypoglycemia which is undesirable. Two patients with a known history of periodic hypoglycemia reported no incidence of it in the three months of post treatment monitoring. Considering patients with pre treatment HbAlc above 7, there was a decrease of 0.36 in 1 month (t stat=2.97, p=0.008) and a decrease of 0.48 in 3 months (t stat=4.86, p=0.0001) which is comparable to the clinical trials for conventional drugs. The decreasing trend is shown in FIG. 4 wherein sample 1 represents the fasting blood sample before the treatment and serves as the baseline. Sample 2 represents post camp fasting blood sample, whereas sample 3 and sample 4 represent fasting blood samples taken after 1 month and after 3 months follow up after of the camp. Mean and standard errors are represented by the bar.
2. Inflammatory markers: The total peripheral blood leucocyte density as well as granulocyte density showed a normalization response (FIGS. 5 and 6). The inflammatory marker C reactive protein (CRP) also showed a normalizing trend (FIG. 7). The six patients that had an alarmingly high pre-treatment level of CRP (>10000 ng/ml) showed a significant decrease in level of CRP by 7365.16 ng/ml in 1 month (t=2.53, p=0.05) and 8388.25 ng/ml in 3 months (t=3.39, p=0.04) as shown in FIG. 8.
3. Endogenous insulin: Fasting plasma insulin showed a negative correlation with HOMA R (FIG. 9)). This implies that there are two types of responses of patients. Those that had low insulin resistance, responded to the treatment by increasing endogenous insulin whereas those with high insulin resistance showed a decrease in insulin as result of decreasing insulin resistance. The mean Homa beta, an index of beta cell function showed a significant increase of 12.21 in 1 month (t=−1.38, p=0.08) and an increase of 18.1 in 3 months which was marginally significant in a one tailed test (t=−1.27, p=0.10) (FIG. 10).
4. Insulin resistance: The Homa R, an index of insulin resistance also showed a significant normalization trend (FIG. 11) indicating that patient with high insulin resistance developed greater sensitivity and those with low initial resistance showed slight increase which was accompanied by increase in endogenous insulin.

### Anecdotal Studies:

1. A 54 year old male was presented with a history of diabetes for six months. Initially he was diagnosed for a high level of insulin resistance and high fasting insulin level. Then he was asked to follow the HBT protocol in accordance with the present invention. The patient did not take any medication prior to or during HBT

The patient followed the HBT protocol rigorously in the 1^{st} week with a compliance level of 80 to 100%. From 2^{nd} to 4^{th} week compliance was poor (<20%). After 4^{th} week compliance was improved and remained consistently between 40 to 60%. The results showed that fasting insulin levels (pre-camp sample 36.2 mlU/L) was dropped dramatically (12 weeks 8.8 mlU/L) without any rise in the fasting sugar which indicates that insulin resistance was reduced. The index HOMA R indicating insulin resistance was decreased from 14.7 to 6.1 within one week. During 2^{nd} to 4^{th} week the progress was slow, however from 5^{th} week again there was rapid reduction in fasting insulin and HOMA R which stabilized after reaching the normal range (2.8). The pre treatment levels of fasting blood sugar (164 mg/dL) and HbAlc (7.1%) were reduced gradually and stabilized close to the normal range (12 weeks sample 120 mg/dL and 6.3% respectively). There was also weight reduction by 2.5 Kg during the first 4 weeks which remained constant thereof.

2. A 71 year old male was presented with a history of diabetes for 4 years. He was under heavy medication including insulin sensitizing drugs, glycosidase inhibitors as well as exogenous insulin. In spite of all the medication and regular exercises, his fasting blood sugar remained consistently above 140 mg/dL and HbAlc above 7% before the treatment with HBT protocol in accordance with the present invention. Then he was asked to follow the HBT regime in accordance with the present invention. The compliance to HBT exercises by him was above 80% in the first week and was consistently above 60% during the follow up period.

In 12 weeks the levels of CRP reduced from 1880 to 409 ng/ml, fasting sugar from 143 to 92 mg/dL and HbAlc from 7.3 to 6.8% and remained stable.

3. A 53 year old female was presented with a history of diabetes for 4.5 years. She was initially diagnosed for fasting blood sugar which was found to be high (exceeding 250 mg/dL.). She was then asked to follow HBT regimen in accordance with the present invention. She followed the HBT regime rigorously for first week, the compliance was between 60 to 80%. Thereafter compliance was poor, i.e. less than 20%. After starting HBT, the baseline HbAlc was declined from 10.2 to 9.3 in 4 weeks and fasting sugar was declined from the base level of 169 to 125 in 4 weeks. Further progress was slow presumably owing to low compliance level but a follow up after 64 weeks revealed fasting sugar as well as HbAlc close to the normal range (110 mg/dL and 7.2% respectively).

4. A 56 year old female was presented with high BMI, hypertension and very high CRP levels although her sugar levels were normal. She was then asked to follow the HBT regime in accordance with the present invention. The compliance by her was moderate to low but consistent over a long period. The CRP levels were monitored periodically over one year. The CRP levels were reduced from >18000 ng/ml to less than 4500 ng/ml in 12 weeks and remained in the range between 2000 to 4000 ng/ml till the end of the monitoring period of one year.

5. A 63 year old male was presented with a history of diabetes for 8 years. He was being treated with metformin before the treatment with HBT protocol in accordance with the present invention. He was then asked to follow the HBT regime He started HBT with good rigour and maintained about 80% compliance for about 4 weeks, which was declined to 40 to 60% in the following 12 weeks and then discontinued. HBT was started again after exactly 1 year and follow up was made for 3 more months.

HbAlc reduction was remarkable during the first 4 weeks (7.7 to 6.6) and CRP was reduced from over 8000 ng/ml to less than 2000 ng/ml in 12 weeks. Later when HBT was almost discontinued, HbAlc increased again to the original levels but CRP level remained low. When HBT regime was started again, there was reduction of HbAlc in 4 weeks which was almost identical in magnitude to the earlier response (7.9 to 6.9) and this level was maintained for the next 8 weeks of monitoring.

### ADVANTAGES OF THE INVENTION

The instant invention offers several benefits as mentioned below:
The holistic behavioral therapy (HBT) simultaneously targets several risk factors and attacks the behavioral root cause behind metabolic syndrome.
Unlike traditional drug treatments which have one way action on a narrow target, HBT has a “normalization” action simultaneously on multiple targets. Drugs are commonly designed to interfere in a specific biochemical or signaling pathway and either always increase or always decrease the end product of that pathway. For example, statins have cholesterol lowering action only. HBT has normalizing action rather than a one way action since it acts by balancing and strengthening the homeostatic mechanisms of brain and endocrine systems of the body. For example it would reduce the blood sugar level if it is abnormally high and increase if it is abnormally low. Therefore it is sensitive to the initial conditions of the patient and exerts appropriate effects accordingly. A normalization effect also means that unlike drugs, any side effects associated with overdose of over-action are unlikely to be observed.
The HBT as described in the instant invention is highly efficient in reducing systemic inflammation.
The HBT of the present invention promises a significant rise in insulin secretion in case of patients having low standing insulin resistance and low fasting insulin levels.
The HBT of this invention also provides for a substantial increase in insulin sensitivity in case of patients with high insulin resistance.

While considerable emphasis has been placed herein on the various components of the preferred embodiment, it will be appreciated that many alterations can be made and that many modifications can be made in the preferred embodiment without departing from the principles of the invention. These and other changes in the preferred embodiment as well as other embodiments of the invention will be apparent to those skilled in the art from the disclosure herein, whereby it is to be distinctly understood that the foregoing descriptive matter is to be interpreted merely as illustrative of the invention and not as a limitation.

1. A method of treatment of Insulin Resistance Syndrome (IRS), said method comprising the following steps:
i. increasing secretions of hormones and growth factors by physically aggressive activities;
ii. normalizing monocyte and neutrophil count by cutaneous immuno-sensory stimulation;
iii. stimulating wound healing and angiogenesis mechanisms by physically aggressive activities and skin stimulation exercises;
iv. stimulating insulin secretion by intense mental activity and by increased epidermal growth factor (EGF) production which is stimulated by physically aggressive activities; and
v. decreasing insulin resistance by isolation of the patient in a serene environment.

2. The method of treatment as claimed in claim 1, wherein the hormones stimulated by physically aggressive activities are testosterone, estradiol, dopamine, melatonin and cholecystokinin.

3. The method of treatment as claimed in claim 1, wherein the growth factor is epidermal growth factor (EGF).

4. The method of treatment as claimed in claim 1, wherein the physically aggressive activities involve games and activities which mimic neuromotor actions of stone age hunters while performing acts like hunting, fighting, punching, hard hitting, aiming, throwing, grabbing, snatching, dodging, escaping, chasing and catching.

5. The method of treatment as claimed in claim 1, wherein the cutaneous immuno-sensory stimulation is induced by pricking, scratching, pinching, and bruising without causing overt injuries.
